(19) United States
(12) Patent Application Publication
(10) Pub. No.: US 2002/0132858 A1
(43) Pub. Date: Sep. 19, 2002

(54) Method for treating sexual disorders

(75) Inventors: Outi Maki-Ikola, Turku (FI); Antti Haapalinna, Turku (FI)

Correspondence Address: FINNEGAN, HENDERSON, FARABOW, GARRETT &, DUNNER LLP, 1300 I STREET, NW, WASHINGTON, DC, 20005, US

(73) Assignee: Orion Corporation

(21) Appl. No.: 10/145,045

(22) Filed: May 15, 2002

Related U.S. Application Data
(63) Continuation of application No. 09/765,340, filed on Jan. 22, 2001.

(51) Int. Cl. A61K 31/137

(52) U.S. Cl. 514/651

(57) ABSTRACT

A method of treating sexual disorders in a mammal by administering to the mammal an effective amount of 1,7,7-trimethylbicyclo[2.2.1]heptane derivative of Formula I

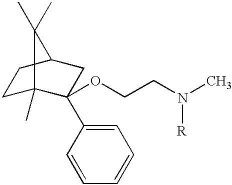

wherein R is hydrogen or methyl, or a pharmaceutically acceptable salt thereof.

### FIELD OF THE INVENTION

The present invention relates in general to a method for treating sexual disorders in a mammal. More particularly, the invention relates to a method for treating sexual disorders in a mammal by administering to the mammal an effective amount of 1,7,7-trimethylbicyclo[2.2.1]heptane derivative of Formula I

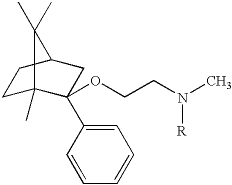

wherein R is hydrogen or methyl, or a pharmaceutically acceptable salt thereof.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

### BACKGROUND OF THE INVENTION

The active ingredients of this invention, (1R,2S,4R)-(−)-2-phenyl 2-(dimethylaminoethoxy)-1,7,7-trimethyl-bicyclo[2.2.1]heptane, known as deramciclane, and (1R,2S,4R)-(−)-2-phenyl-2-(methylaminoethoxy)-1,7,7-trimethyl-bicyclo[2.2.1]heptane, known as N-desmethylderamciclane, and their pharmaceutically acceptable acid addition salts with inorganic and organic acids generally used for the purpose, fall within the disclosures of U.S. Pat. No. 4,342,762 and International Patent Application No. WO 98/17230, respectively, which are both incorporated herein by reference.

These compounds are selective serotonin 5HT2A- and/or 5HT2C-receptor antagonists. They have shown anxiolytic-like effects in animal test models.

### DESCRIPTION OF THE INVENTION

Applicants have surprisingly discovered that the compounds of Formula (I) have therapeutic effect on sexual disorders in a mammal. Accordingly, an object of the present invention is a method for treating sexual disorders in a mammal by administering to the mammal an effective amount of a compound of Formula (I) or a pharmaceutically acceptable salt thereof.

Sexual disorders, such as male sexual dysfunction or female sexual dysfunction, are often chronic in nature. Male sexual dysfunction includes sexual desire disorders, sexual arousal disorders (erectile dysfunction), orgasm disorders (premature ejaculation or inhibited orgasm) and sexual pain disorders. Female sexual dysfunction includes decreased sexual desire (libido), decreased sexual arousal, dyspareunia (pain during intercourse) and diminished ability to achieve orgasm.

Sexual disorders may be caused directly or indirectly by several diseases or conditions, such as various somatic (e.g., neurological or vascular) diseases, and psychiatric disorders. Psychiatric disorders include but are not limited to affective disorders, such as depression or anxiety disorders, such as Generalized Anxiety Disorder (GAD), Social Anxiety Disorder (SAD), Panic Disorder (PD), Obsessive Compulsive Disorder (OCD), Post-Traumatic Stress Disorder (PTSD) or agoraphobia. In addition, various drugs used for the treatment of psychiatric disorders, for example anxiolytic compounds like buspirone and benzodiazepines such as diazepam, alprazolam or lorazepam, antidepressants such as venlafaxine and selective serotonin reuptake inhibitors (SSRIs), known to be effective also in GAD, and antipsychotics, frequently cause sexual disorders such as decreased libido as an adverse effect.

For the purposes of this disclosure and claims the term “treatment” means treatment in order to cure or alleviate the disorder or its symptoms, and to treatment in order to prevent the development or the exacerbation of the disorder or its symptoms.

Pharmaceutically acceptable salts of the compound of Formula (I) can be formed with inorganic acids, e.g. hydrohalogenic acid such as hydrochloric acid or hydrobromic acid, sulfric acid, phosphoric acid or nitric acid, or organic acids e.g., tartaric acid, succinic acid, malic acid, maleic acid, fumaric acid, citric acid, or lactic acid. Salt with fumaric acid is preferred.

Pharmaceutical compositions containing a compound of Formula (I) or a pharmaceutically acceptable salt thereof as the active ingredient include the usual oral dosage forms, such as tablets, capsules, and liquid preparations. In oral dosage forms, the active ingredient can be mixed with suitable pharmaceutically acceptable excipients, such as starch, lactose, sucrose and magnesium stearate, in accordance with conventional pharmaceutical practice.

The precise amount of the drug to be administered to a mammal for the treatment of sexual disorders is dependent on numerous factors known to one skilled in the art, such as the compound to be administered, the general condition of the patient, the condition to be treated etc. For example, the usual recommended oral daily dose of deramciclane would be about 5-150 mg/day, or about 10-60 mg/day, or about 30-60 mg/day, or about 30 mg/day.

The invention will be further clarified by the following example, which is intended to be purely exemplary of the invention.

### EXAMPLE

The effects of deramciclane were studied in a randomised placebo-controlled double-blind study. The subjects were randomly assigned to four parallel groups to receive one tablet twice daily (b.i.d) of a placebo, 5 mg (=10 mg/day), 15 mg (=30 mg/day), or 30 mg (=60 mg/day) deramciclane.

When analyzing the number of patients with decreased libido, 2 out of 51 patients on placebo experienced it, but none of the patients treated with deramciclane 15 mg b.i.d. (n=53) or 30 mg b.i.d. (n=54) experienced decreased libido (p=0.04, Chi-square-test). In addition, when the Udvalg for Kliniske Undersogelser (UKU) scale was used, 21% of 53 patients experienced orgastic dysfunction at the baseline but only 12% of them after 8-week treatment with deramciclane 15 mg b.i.d.

Although the invention has been illustrated by the preceding example, it is not to be construed as being limited to the materials employed therein; rather, the invention is directed to the generic area as herein disclosed. Various modifications and embodiments thereof can be made without departing from the spirit or scope thereof.

We claim:

1. A method of treating a sexual disorder in a mammal, comprising administering to said mammal an effective amount of at least one compound of Formula (I) 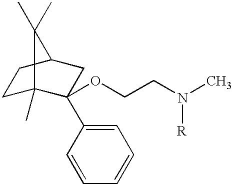
wherein R is hydrogen or methyl, or a pharmaceutically acceptable salt of the compound of Formula (I).

2. The method of claim 1, wherein the sexual disorder is male sexual dysfunction.

3. The method of claim 1, wherein is the sexual disorder is female sexual dysfunction.

4. The method of claim 1, wherein the sexual disorder is associated with an affective disorder.

5. The method of claim 4, wherein the affective disorder is depression.

6. The method of claim 1, wherein the sexual disorder is associated with an anxiety disorder.

7. The method of claim 6, wherein the anxiety disorder is General Anxiety Disorder.

8. The method of claim 6, wherein the anxiety disorder is Social Anxiety Disorder.

9. The method of claim 6, wherein the anxiety disorder is agoraphobia.

10. The method of claim 6, wherein the anxiety disorder is Panic Disorder.

11. The method of claim 6, wherein the anxiety disorder is Obsessive Compulsive Disorder.

12. The method of claim 6, wherein the anxiety disorder is Post-Traumatic Stress Disorder.

13. The method of claim 1, wherein the sexual disorder is associated with administration of a drug used for the treatment of a psychiatric disorder.

14. The method of claim 1, wherein the mammal is human.

15. The method of claim 1, wherein the at least one compound is deramciclane or a pharmaceutically acceptable salt thereof.

16. The method of claim 1, wherein about 5 to about 150 mg/day of at least one compound as claimed in claim 1 is administered.

17. The method of claim 16, wherein the amount administered is about 10 to about 60 mg/day.

18. The method of claim 17, wherein the amount administered is about 30 mg/day.
